(19) United States
(12) Patent Application Publication
(10) Pub. No.: US 2006/0166270 A1
(43) Pub. Date: Jul. 27, 2006

(54) Detection of demyelination

(75) Inventors: Jacobus Thomas W. E. Vogels, Utrecht (NL); Lybert Adriaan 'T Hart, Utrecht (NL)

Correspondence Address: NORRIS, MCLAUGHLIN & MARCUS, P.A., 875 THIRD AVE, 18TH FLOOR, NEW YORK, NY, 10022, US

(73) Assignee: NEDERLANDSE ORGANISATIE voortoegepastnatuurweten schappelijk ONDERZOEK TNO, DELFT (NL)

(21) Appl. No.: 10/534,519

(22) PCT Filed: Nov. 10, 2003
(86) PCT No.: PCT/NL03/00785

§ 371 Date: Feb. 28, 2006

(30) Foreign Application Priority Data
Nov. 11, 2002 (NL) 1021892

(51) Int. Cl. G01N 33/53; G01N 33/92

(52) U.S. Cl. 435/7.1; 436/71

(57) ABSTRACT

The present invention relates to a method for the detection of demyelinization in a mammal, comprising providing an NMR spectrum of metabolites in a body fluid of an individual of said mammal in which demyelinization is suspected and comparing said NMR spectrum with a difference profile, comprising a plurality of NMR spectral line positions, which express the normalized difference between one or more NMR spectra of metabolites in a body fluid of one or more healthy individuals of said mammal, and one or more corresponding NMR spectra of metabolites in a corresponding body fluid of one or more individuals of said mammal in which demyelinization has been diagnosed. The invention further relates to the said difference profile for the detection of demyelinization in a mammal and to methods for the manufacture thereof. The invention also relates to a biomarker for detection of demyelinization, in particular multiple sclerosis, characterized in that the biomarker is a polar head group of phosphoglyceride and to the use of that biomarker in detection of demyelinization and multiple sclerosis.

The invention relates to biomarkers for detection of demyelinization in a mammal, to a difference profile between NMR spectra as a metabolite pattern for determination of demyelinization, to a method for manufacturing such a difference profile and to a method for detection of demyelinization in a mammal by means of a biomarker and/or difference profile according to the invention.

In the central nervous system (CNS), the axons are surrounded by a myelin sheath, which is produced by oligodendrocytes. The insulating function of the myelin sheath is important for the stimulus conduction of action potentials along the axons. Multiple sclerosis (MS) is a chronic disease of the CNS in which the protective myelin sheath of the nerve fibers is affected and decomposed (demyelinization). This myelin degradation results in a (temporary) interruption of the nerve impulses and therefore greatly affects the motor system, vision, sense, etc. of the MS patient. The sclerosis of affected nerve tissue may eventually lead to permanent paralysis.

MS is the most common inflammatory disease of the central nervous system and chiefly affects young adults. The disease has various forms of progression, with the relapsing-remitting form (40% of patients) and the secondary progressive form (40% of patients) occurring the most.

By now, it is known that the relapsing-remitting form, in which attack and recovery alternate, can result in the more severe secondary progressive form. Research has shown that this group of patients greatly benefits from slowing down the disease process by means of therapy to postpone the transition period as long as possible. Currently, such a therapy involves medication with interferon-beta or glatiramer acetate (copolymer-1).

In the secondary progressive manifestation, the clinical picture no longer involves any recovery moments and the symptoms only increase in number and extent. Also for this group of patients, medication is very useful to slow down the progression of the disease.

At present, there is no diagnostic test (laboratory detection or measuring technique) for MS by means of which the disease can be diagnosed with 100% certainty. The diagnosis of MS is complex and is made on the basis of a neurological examination (motor system, coordination, sense and reflexes) in combination with examination on indicative proteins in the lumbar fluid (liquor cerebrospinalis) via a lumbar puncture and MRI (magnetic resonance imaging) examination for the occurrence of sites of inflammation and lesions in brains or spinal cord. This combination is important because, in many cases, clinical examination shows a completely normal picture; while the patient already has many symptoms. All the same, the neurologist will not always be able to diagnose MS with certainty. Therefore, for an actual diagnosis, it is also necessary to demonstrate progression of the clinical picture in time.

A problem in examination for MS is that the actual progress of the disease is difficult to determine. Despite the fact that MRI examination is very important, this method has the disadvantage that it is not possible to determine whether a site of inflammation, which is visible on a MRI scan indeed involves tissue degradation. The relationship between the severity of the disease and the number or the nature of the visible sites of inflammation is too slight for this.

Therefore, by means of the current techniques, the extent and rapidity of tissue degeneration in the central nervous system cannot be determined or cannot simply be determined. This makes it impossible to start the therapeutic treatment already at an early stage, so that the disease is often already at an advanced stage even before medication is administered.

Also, the absence of early diagnostics limits the development of more specific and more effective therapies. There is a great need for alternative methods which can demonstrate quantitatively, reliably, sensitively and specifically demonstrate demyelinization-related changes in the central nervous system. Further, there is a need for a method by means of which demyelinization can be diagnosed at an early stage, preferably before the process has led to irreversible changes.

The use of molecular markers (or biomarkers) which are specific for demyelinization could fulfill these needs and can make an important contribution to diagnosis, prognosis, and monitoring of the progress of the disease. Further, by means of such molecular markers, research into the effect of clinical treatment therapies and the development of new medicines could be facilitated. Thus, molecular markers are considered crucial for effectively carrying out preclinical studies (both in vitro and in vivo in laboratory animals) and studies directed at the pathophysiology of demyelinization in general and MS in particular.

An ideal molecular marker is disease-specific, reflects the actual disease activity, can be used for determining the effectiveness of therapy and contributes to the reliable prognosis of the disease. However, all these requirements do not need to be integrated in one single marker; a combination of complementary markers is possible and could, in particular cases, perform even better.

Markers which are currently used in MS-related research comprise immunological markers such as free light chains of immunoglobulin G, cytokines and cytokine receptors, oligoclonal bands of antibodies, antiviral antibodies, intrathecal immunoglobulin production, T-cells, adhesion molecules and other surface molecules. Immunological markers are determined in blood or lumbar fluid. The disadvantage of such immunological markers is that, while being characteristic of, they are not unique to MS.

Other biomarkers used in MS research are markers for tissue of the central nervous system. These include inter alia Myelin Basic Protein (MBP), S-100 protein (Missler et al. 1997. Acta Neurol. Scand. 96:142-4), neuron specific enolase (NSE) (Persson et al. 1987. Stroke 18:911-8), glial fibrillary acidic protein (GFAP) (Eng et al. 2000. Neurochem. Res. 25:1439-51), neurofilaments (Rosengren et al. 1996. J. Neurochem. 67:2013-8), adhesion molecules of nerve cells (Elovaara et al. 2000. Arch. Neurol. 57:546-51), and ciliary neurotrophic factor (CNTF) (Massaro. 1998. Mult. Scler. 4:228-31). These tissue markers are characteristic of tissue damage and are therefore actually no direct indication of demyelinization.

Finally, biomarkers such as, for instance, gliotoxin (Menard et al. 1998. J. Neurol. Sci. 154:209-21), neopterin (Sorensen. 1999. Mult. Scler. 5:287-90) and matrix metalloproteinases (Sellebjerg et al. 2000. J. Neuroimmunol. 102:98-106) are used. However, such markers are not direct markers of the demyelinization process either. For a more detailed description of Multiple Sclerosis and aspects of its diagnostics, reference is made to the abovementioned publications, as well as the overview publication Multiple Sclerosis: Current Status and Strategies for the Future. Joy & Johnston, eds. Nat. Acad. Press, Washington D.C., 2001 and references therein.

It is an object of the present invention to provide new systems and methods for the detection of demyelinization.

Another object of the present invention is to provide systems and methods which solve at least some of the problems associated with existing systems and methods for the detection of demyelinization as described hereinabove.

Another object of the present invention resides in providing systems and methods as described hereinabove which can be used in in vivo and/or in vitro medical diagnostics.

It has now been found that, in the urine of an individual with demyelinization, metabolites are present which are not, or in larger or smaller quantities, present in healthy individuals. It was possible to demonstrate the presence of these demyelinization-specific metabolite concentrations by means of a proton nuclear magnetic resonance (¹H NMR) spectroscopic analysis of the metabolites in the urine of mammals. Therefore, these metabolites can be used, individually or in combination, as a biomarker in the diagnostics and prognostics of demyelinization.

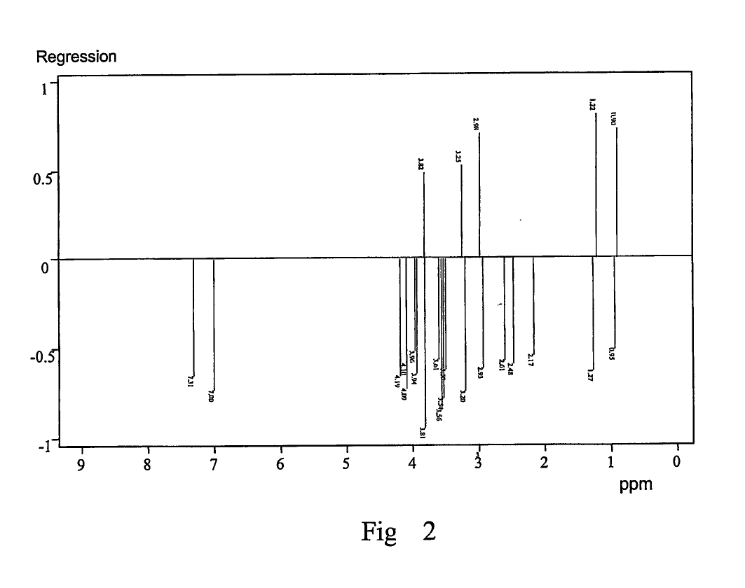

It has further been found that a collection of statistically significant differences between the signal intensity of a large number of spectral lines with defined positions in the NMR spectrum, recorded from metabolites in the urine of a healthy individual, and the signal intensity of corresponding spectral lines in the NMR spectrum recorded from metabolites in the urine of an individual with demyelinization can provide a pattern which facilitates the detection of demyelinization. In the present invention, this pattern is referred to as a difference profile or metabolic fingerprint. Such a difference profile can be graphically represented as a factor spectrum (see FIG. 2).

The present invention therefore relates to a difference profile for the detection of demyelinization in a mammal, comprising a plurality of spectral line positions and, optionally, corresponding signal intensities of NMR spectral lines, which express the normalized difference between one or more NMR spectra of metabolites in a body fluid of one or more healthy individuals of this mammal, and one or more corresponding NMR spectra of metabolites in a corresponding body fluid of one or more individuals of this mammal in which demyelinization has already been diagnosed.

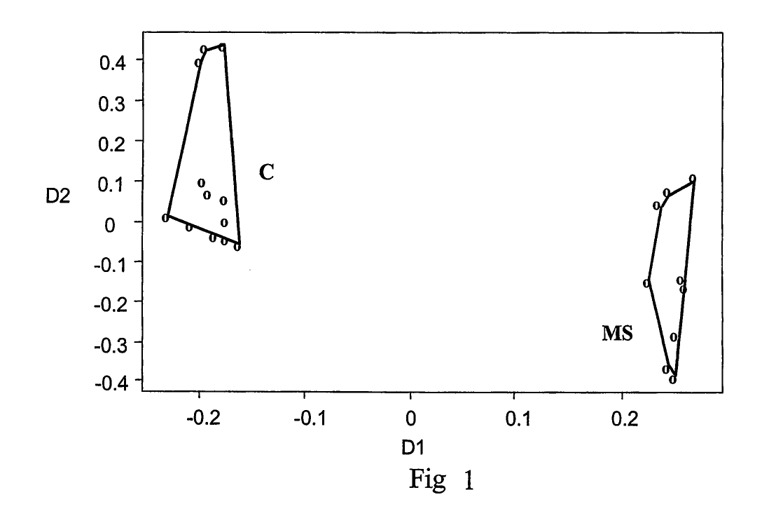

FIG. 1 is a representation of a score plot of NMR spectra obtained in the manner as described in the description below and Example 1. On the horizontal axis, component D1 (100.00%) is plotted. On the vertical axis, component D2 (0.00%) is plotted. The left uninterruptedly outlined cluster (A) is a cluster of NMR spectra of healthy control individuals, while the right interruptedly outlined cluster (B) represents a cluster of NMR spectra of patients with demyelinization.

FIG. 2 is a representation of a factor spectrum (also difference profile or metabolic fingerprint) of demyelinization obtained in the manner as described in the description below and Example 1. On the horizontal axis, the spectral line position is plotted in “ppm”. On the vertical axis, the signal intensity is plotted in “Regression”.

In the present invention, a difference profile is defined as a characteristic selection of NMR spectral lines with defined positions whose signal intensity significantly differs in normalized NMR spectra of metabolites in a body fluid of demyelinization patients compared to normalized NMR spectra of metabolites in a body fluid of healthy individuals. Such a difference profile comprises the spectral line positions and optionally their corresponding signal intensities.

In the present invention, a normalized NMR spectrum is defined as an NMR spectrum in which the diversity or variation in the signal intensities of the spectral lines between samples is limited by arithmetically taking glitches into account. For normalization, the sum of the squares of all intensities is equated with 1. The reason for this is that it is assumed that each sample comprises an equal amount of information. By carrying out normalization, the absolute amount of information in each NMR spectrum is equated (equal surfaces under the NMR spectra), so that they become mutually comparable.

A changing signal intensity of a particular spectral line in two comparable NMR spectra indicates that the concentration of hydrogen atoms in one of those samples has changed and that, thus, the amount of one or more chemical components containing these atoms, in this case metabolites, has changed in one of those samples.

So, a difference profile according to the invention comprises a collection of spectral line positions in a normalized NMR spectrum whose corresponding signal intensity is increased or decreased due to demyelinization compared to the signal intensity of corresponding spectral line positions in a normalized NMR spectrum of healthy individuals.

Preferably, a difference profile according to the invention comprises spectral line positions whose corresponding signal intensities are increased and/or decreased by a particular factor in the spectrum of a demyelinization patient in relation to a corresponding spectrum of a healthy individual. This factor can be used for applying a (positive) threshold value (or reference value) for increases and a corresponding (negative) threshold value for decreases. Spectral line positions whose corresponding signal intensities are above or below the corresponding threshold value are included in the difference profile. The endogenous and exogenous metabolites (see below) have been eliminated from such a difference profile so that the data are reduced to specific and “significant” demyelinization-related changes.

For eliminating endogenous and exogenous metabolites from a difference profile according to the invention, a threshold value which corresponds to approximately one and a half times, preferably approximately two times, more preferably approximately three times the signal to noise ratio can very suitably be used in the normalized spectrum. Here, noise in the NMR spectrum is understood to mean the signals coming from aspecific measurement events, such as for instance machine noise, environmental fluctuations, and/or contaminations in the chemicals.

It is also possible to use, for instance, the value of the average signal intensity of 60-99%, preferably 70-95%, more preferably 80-90% of all spectral line positions showing a change in intensity between healthy individuals and demyelinization patients as a threshold value for obtaining a difference profile according to the invention.

The choice for the level of the threshold value will also inter alia depend on the individual properties of the mammal for which the difference profile is determined. Such properties comprise sex, age, stage of life (fertile/infertile), diet, possible medication, genetic background, and, in humans, tobacco and/or alcohol consumption. The use of homogeneous groups of individuals is preferred in the methods according to the invention described hereinbelow, with a homogeneous group being defined as a group of individuals with as many comparable properties as possible, the only difference being the presence or absence of the disease.

Preferably, a normalized spectrum of metabolites in a body fluid of a mammal comprises a set of data coming from a homogeneous group of individuals. That means that a difference profile according to the invention for detection of demyelinization in a male individual comprises NMR spectral line positions with corresponding signal intensities of preferably exclusively male individuals. A difference profile for demyelinization can therefore be different depending on the properties of the individuals from which it has been obtained.

Preferably, a normalized spectrum of metabolites in a body fluid of a mammal represents a set of data coming from at least two, more preferably at least three, still more preferably at least four, and even more preferably at least five individuals.

A difference profile can very suitably comprise 3 to 1,000 spectral line positions corresponding to possibly original spectral lines. Preferably, a difference profile according to the invention comprises 10 to 500, more preferably 15 to 100, and still more preferably 20 to 70 spectral line positions. Very good results have been obtained with a difference profile comprising 30 to 50 spectral line positions.

The number of spectral line positions from which the difference profile is built up is chiefly determined by the definition of the threshold value mentioned. This threshold value, in which the value for the pitch of the noise in the normalized spectra can have been taken into account, indicates from which value differences in the height of a spectral line between individuals in which demyelinization has been diagnosed and healthy individuals are “significant”. A difference in height can be either positive (increase of intensity) or negative (decrease of intensity).

As said, the detection of demyelinization by means of a difference profile according to the invention is preferably used in individuals with properties which are corresponding or similar to those of individuals from which the difference profile has been obtained, but this is by no means necessary.

The present invention also relates to a method for manufacturing a difference profile for the detection of demyelinization in a mammal.

A difference profile according to the invention can very suitably be manufactured by means of a method comprising the step of providing a first set of positions and corresponding intensities of spectral lines in an NMR spectrum which has been recorded from metabolites in a body fluid of healthy individuals of a mammal.

As a body fluid which can be used in a method according to the invention, in principle, any body fluid can be used. Preferably, a body fluid is used which can be obtained in a non-invasive manner. It is most preferred that the body fluid be urine.

Although, in embodiments of the present invention, in principle, different measurement methods for measuring metabolites in a body fluid can be used, preferably proton nuclear magnetic resonance spectroscopy is used. An NMR instrument with a frequency of at least approximately 200 MHz is, in principle, suitable, but there is a preference for use of instruments with a higher frequency, such as at least approximately 300 MHz, more preferably at least approximately 400-600 MHz.

For carrying out NMR spectroscopic analysis, samples of a body fluid can very suitably be lyophilized and the lyophilisate can then be reconstituted in a suitable buffer, for instance a sodium phosphate buffer, which is prepared on the basis of D₂O. A suitable acid content for such a buffer is in the range of pH 4-10, preferably of pH 4-8, and more preferably, such a buffer has a pH of approximately 6. Preferably, different samples which will be mutually compared are reconstituted in buffers of equal pH. The reconstitution of the lyophilized components of a sample of a body fluid in a buffer of equal pH serves to minimize spectral differences caused by differences in pH between different samples. To the reconstituted sample, further, an internal standard, such as for instance TMSP (sodium trimethylsilyl-[2,2,3,3,-²H₄]-1-propionate) or tetramethylsilane can be added. Then, an NMR spectrum can be recorded from these samples, the NMR instrument being set for ¹H NMR analysis. Preferably, an NMR spectrum of a sample is recorded in triplicate. In general, default settings as recommended by the manufacturer can be used for this purpose. The measurement results are shown in chemical shift in relation to the internal standard and are expressed in “ppm” (parts per million). In the present invention, a spectral line position is expressed in “ppm”, while the signal intensity is expressed in “regression” (see also FIG. 2), as is conventional in the field.

To the recorded spectra, optionally, a manual baseline correction is applied and the spectra are then processed into so-called line listings by means of standard NMR procedures. For this purpose, all lines in the spectra above the noise are collected and converted into a data file which is suitable for multivariate data analysis.

Preferably, several healthy individuals of the respective mammal are measured so that glitches can be arithmetically taken into account. Such an arithmetic account of glitches can very suitably take place in combination with the process of normalization of the measurement data. For determining a normalized spectrum of metabolites in a body fluid of a healthy mammal, in principle, one single healthy individual can be measured, but preferably, spectra coming from a group of healthy individuals are used, more preferably a homogeneous group.

Normalization of several recorded NMR spectra contributes to the reliability of a set of values obtained from a plurality of individuals. Further, normalization allows the comparison of a separately recorded spectrum with a set of previously recorded spectra.

A method for manufacturing a difference profile also comprises the step of providing a second set of positions and corresponding signal intensities of spectral lines in an NMR spectrum which has been recorded in a corresponding manner from metabolites in a corresponding body fluid of individuals of that same mammal in which demyelinization has been diagnosed.

Preferably, here as well, several individuals of a homogeneous group of the respective mammal in which demyelinization has been diagnosed are measured so that glitches can be arithmetically taken into account. To the recorded spectra, optionally, a manual baseline correction is applied and the spectra are then processed into so-called line listings by means of standard NMR procedures. For this purpose, all lines in the spectra above the noise are collected and converted into a data file which is suitable for multivariate data analysis. The recorded NMR spectra are preferably normalized in the above-described manner.

Finally, a method for manufacturing a difference profile comprises the step of comparing the normalized values of the first and second set of positions and corresponding intensities of spectral lines in an NMR spectrum, and detecting the differences between them for obtaining a difference profile according to the invention.

Multivariate data analysis or pattern recognition can very suitably be used to visualize differences related to disease and treatment in these spectra. The arithmetic method based on the Partial-Linear-Fit algorithm as described in WO 02/13228 is particularly preferred. This algorithm enables adjustment of small variations in the position of the spectral line in NMR spectra without loss of resolution.

The above-described Partial-Linear-Fit algorithm comprises a principal component discriminant analysis (PCDA) part. Here, the number of variables is first reduced by means of principal component analysis (PCA). The projections, so-called scores, of samples on the first principal components (PCs) are used as a starting point for linear discriminant analysis. The scores of the samples are plotted in a score plot, where similar samples tend to cluster and dissimilar samples will be spaced a larger distance from each other (see FIG. 1). The relation of discriminant axes to the original variables (NMR signals) is visualized in a loading plot. Here, the position of the original variables is shown so that the length of the variable vector parallel to a discriminant axis is proportional to the loading of that variable to that axis.

Another possibility to visualize the data is by means of factor spectra (see inter alia FIG. 2), which correlate to the positions of clusters in score plots (e.g. the demyelinization cluster in FIG. 1) by graphical rotation of loading vectors. These factor spectra, or metabolic fingerprints, made in the direction of maximum separation of one category in relation to another category, provide insight in the types of metabolites responsible for separation between the categories.

Therefore, a difference profile according to the present invention can very suitably be shown as a factor spectrum, an example of which is shown in FIG. 2, or as a table with spectral line positions, an example of which is shown in Table 1.

Since, in the present invention, the analytical methodology of proton nuclear magnetic resonance spectroscopy is used for obtaining numeric data concerning metabolites, the values obtained depend on the settings of the instrument and the conditions under which the measurement is carried out. Also, the absolute values depend on the reference (e.g. the internal standard) used in the measurement. A difference profile, as it is shown in Table 1, thus comprises values which can differ between different measurement moments and between different measurement conditions. For this reason, the values as shown in Table 1 are not absolute values. The meaning of the individual values of both the spectral line positions and the possible spectral line intensities in the difference profile for demyelinization thus substantially resides in their ratio and position in relation to each other and therefore in the pattern of these values.

Due to deviant measurement conditions as indicated hereinabove, the ppm value of a spectral line defined in Table 1 can be located at a point with a ppm value of ±0.05 ppm as shown in Table 1.

The present invention further relates to a method for the detection of demyelinization in a mammal, comprising the steps of providing an NMR spectrum of metabolites in a body fluid of an individual of this mammal in which demyelinization is suspected and comparing this NMR spectrum with a difference profile determined according to the invention for a corresponding body fluid in a corresponding mammal. Such a comparison step can be carried out visually, but also arithmetically.

It is possible, but not necessary, to normalize the NMR spectrum of metabolites in a body fluid of an individual of this mammal in which demyelinization is suspected prior to comparing it with a difference profile according to the invention by means of spectra of metabolites in a body fluid of healthy individuals of the respective mammal. If it appears from the comparison step that the characteristic difference profile is really comprised in the spectrum recorded from an individual in which demyelinization is suspected, the presence of the disease is thus determined.

It is also possible to plot the data of the spectrum recorded from an individual in which demyelinization is suspected in a score plot, such as for instance the score plot of FIG. 1, and to determine whether the data fall within the cluster of “demyelinization” spectra. If these data of an individual in which demyelinization is suspected do not fall within the cluster designated “demyelinization”, the disease is not present in the individual. In the present invention, such a diagnostic method step is understood to be comprised in the step for comparing an NMR spectrum with a difference profile.

Endogenous metabolites are formed in the body by metabolic conversion processes and travel via blood vessels or lymphatic vessels. Exogenous metabolites originate outside the body, e.g. in the form of medicines.

Metabolites are waste products found in the body in different forms and numbers. For instance, in a healthy body, the ratio and the occurrence of metabolites in a body fluid, such as urine or blood, are totally different than in an unhealthy body.

In principle, by means of biomarkers, it is possible to quickly distinguish the unhealthy condition from a healthy condition. In the present context, a biomarker is understood to mean an organic compound or its metabolite, or specific patterns or specific amounts of several organic compounds or their metabolites, which can be found in the body of a mammal and which is/are the result of a subclinical or clinical event in that body.

The present invention provides a method for the identification of a biomarker for demyelinization, comprising manufacturing a difference profile according to the invention and identifying a metabolite characterized by one or more defined spectral lines in this difference profile.

The identification of a metabolite which is characterized by one or more defined spectral lines in a difference profile can, for instance, be done by coupling a mass spectrometer to the NMR instrument and analyzing the metabolite corresponding to one or more defined spectral lines by means of mass spectrometry (MS). A skilled person is familiar with mass spectrometry for the identification of substances and metabolites. However, determining the identity of a metabolite corresponding to one or more defined spectral lines can also be done by recording the NMR spectrum from known metabolites and comparing it to the NMR spectral lines in a difference profile according to the invention.

It could be determined that a difference profile for demyelinization according to the invention, as shown in FIG. 2 and Table 1, contains spectral lines with a positive regression (i.e. spectral lines whose height has increased) which are characteristic of polar head groups of lipids which are related to phosphoglyceride or polar head groups of phosphoglycerides themselves. It is assumed that, as a result of the demyelinization and the accompanying complex degradation and inflammatory symptoms, these metabolites are excreted in the urine and that, thus, the excretion of these metabolites in the urine is specific for the presence of demyelinization.

It is known that myelin consists of 70% of lipids and 15-30% of proteins and contributes to a great extent to the total lipid content of the white mass. Although there are no lipids that are unique to the white mass of the central nervous system, the quantitative lipid composition of white and gray mass differs considerably.

The lipid fraction of human myelin contains 22% of cholesterol, 15% of phosphatidylethanolamine, 9% of phosphatidylserine, 10% of phosphatidylcholine, 8% of sphingomyelin, 28% of glycolipids (mainly galactocerebroside), and 8% of other lipids. Axonal membranes contain a specific type of phosphoglyceride, namely plasmalogens. Because the biochemical composition of brain myelin of all mammals corresponds to a great extent, it is likely that the same values apply to animal species such as simians, guinea pigs and rodents.

Plasmalogens are phosphoglyceride analogs with ethanolamine as the most usual polar head group and with less choline than phosphoglyceride.

It is assumed that, due to their limited size, the released polar head groups of phosphoglycerides, such as phosphatidylethanolamine, phosphatidylcholine, phosphatidylserine or phosphatidylinositol, are rapidly released from the lesions, are secreted in the body fluid and excreted via the urine without substantial metabolic modification and proportionally to the demyelinization activity. It is not known whether the polar head group molecules are excreted in the urine in free condition or in a derived form, for instance conjugated to sulphate or phosphate.

The abovementioned metabolites are found in increased amounts in the urine of demyelinization patients, and are therefore eminently suitable to be used as biomarkers. Metabolites which decrease in amount can less well be applied as biomarkers due to the danger of false negative results in particular detection methods.

Further, by means of factor spectrum analysis, it could be determined that, as a result of multiple sclerosis, a number of specific spectral lines show an increase in intensity corresponding to characteristic metabolites. The ¹H chemical shifts of these characteristic metabolites are shown in Table 2 and the metabolites corresponding to these spectral lines were identified as: N-acetylaspartate (singlet at 2.05 ppm [assignment CH3] and multiplet at 2.91 and 1.95 ppm [CH2]); inositol (doublet doublet at 3.25 ppm [H1/H3] and triplet at 4.10 ppm [H2]); choline (multiplet at 3.19 ppm [NCH2] and multiplet at 3.94 ppm. [OCH2]); neopterin (multiplet at 4.34 and 4.44 ppm [CH2], and multiplet at 4.60 and 4.70 ppm [CH], and singlet at 5.20 ppm [OCH2]), and taurine (triplet at 3.26 ppm [CH2SO3] and 3.31 ppm [NCH2]). Individually or in combination, these metabolites can very suitably be used as biomarkers according to the present invention for detecting multiple sclerosis in a patient, where increases in the concentration of the biomarkers indicate, for instance, the (increased) degradation or conversion of the base material from which these metabolites originate.

It is assumed that these metabolites are excreted in the urine as a result of the disease, and the accompanying complex physiological degradation and inflammatory symptoms, and that thus, the excretion of these metabolites in the urine is specific for the presence of the disease.

Therefore, metabolites with a positive regression in a difference profile according to the invention can very suitably be used as a biomarker in a system for the rapid and early detection of demyelinization. In many cases, it will not be possible to conclude from the difference profile whether the metabolites are excreted in the urine in a free condition or in a derived form, for instance conjugated or bound in another manner. For instance, polar head groups can be bound to glycerol. However, a skilled person will understand that the metabolites described can be used as biomarkers in any condition in which they may be found in the body fluid.

Therefore, the invention also relates to a biomarker for diagnosis and prognosis of demyelinization in general and multiple sclerosis in particular, characterized in that the biomarker is a polar head group of phosphoglyceride.

A biomarker according to the invention can be chosen from the group consisting of phosphatidylethanolamine, phosphatidylcholine, phosphatidylserine and/or phosphatidylinositol, parts or derivates thereof.

The present invention further relates to a method for the detection (i.e. the diagnosis and/or prognosis) of demyelinization in a mammal, comprising measuring a biomarker according to the invention in a body fluid, preferably urine. Such a measurement preferably comprises the detection, in a body fluid of an individual of a mammal in which demyelinization is suspected, of a quantitative change in the occurrence of a biomarker in relation to a normal value for that biomarker which is found in a body fluid of healthy individuals and which quantitative change corresponds to the regression of that biomarker in the difference profile for demyelinization.

A measurement of a biomarker can also comprise the detection of a pattern of concentrations or amounts of metabolites in a body fluid of an individual of a mammal in which demyelinization is suspected in the case that the biomarker is a pattern of several metabolite concentrations. If such a pattern of concentrations or amounts of metabolites, which pattern is measured in the form of a biomarker measurement in an individual of a mammal, corresponds to the difference profile of the respective disease for which the biomarker has been determined, the disease is present in that individual. In that case, a qualitative biomarker measurement is involved.

So, a method for detection of demyelinization in a mammal according to the invention comprises the quantitative or qualitative detection of a biomarker according to the invention in a body fluid of that individual.

A measurement of a biomarker according to the invention is preferably carried out for urine.

A measurement of a biomarker in a body fluid of an individual of a mammal for the detection of demyelinization according to the invention will always comprise the step of comparing the measurement value found to a reference, which reference can comprise a characteristic value for healthy individuals and/or a characteristic value for individuals in which demyelinization has been diagnosed.

A diagnosis can be made on the basis of the results of the measurement of a biomarker according to the invention. For instance, a normal level of metabolites or a normal pattern of metabolites will provide the diagnosis “healthy”. Conversely, a deviating metabolite pattern or a deviating metabolite level will provide the diagnosis “demyelinization”.

By means of the present invention, it is therefore possible to detect demyelinization in a mammal by observing specific biochemical changes in the body fluid of an individual of a mammal, which changes are preferably detected by measurement of a biomarker according to the invention.

This biomarker can be detected in a body fluid in different manners. For instance, NMR and/or Mass Spectrometry (MS) can be applied to a sample of a body fluid.

An even simpler and more rapid diagnosis can be made by using microsystem technologies, for instance a “microfluidics” instrument in combination with specific fluorescent enzymes by means of which the metabolites found in the samples to be tested can be quantitatively and qualitatively measured. A skilled person will be able, without many problems, to acquaint himself with the state of the art in the area of the rapid detection of metabolites in order to formulate methods for detecting biomarkers according to the present invention in a body fluid of a mammal for the diagnosis and/or prognosis of demyelinization. (See for instance Microfabrication Technology for Biomedical Innovations. Proc. Cambridge Healthtech Inst. 3rd Annual Conf., October 1997, San Jose, USA).

By means of the present systems and methods, demyelinization can be diagnosed in a quantitative manner. For this purpose, for instance, a database can be compiled of sets of NMR spectra from metabolites in a body fluid of individuals in which demyelinization has been diagnosed, the demyelinization being at different stages of progression and these sets being annotated to quantitative data of the progression of the demyelinization, for instance in combination with MRI or other biomarkers. By formulating difference profiles for demyelinization at different stages of progression, a quantitative series of difference profiles can be obtained. By carrying out a comparison between an NMR spectrum of an individual in which demyelinization is suspected, or of which the severity of the demyelinization is to be determined, and the quantitative series of difference profiles, the presence of demyelinization can be quantitatively expressed. Further, the progression of the disease can be quantitatively followed in this manner.

It is also possible to use a biomarker according to the invention for quantitative analysis of demyelinization. As described above, such an analysis comprises the quantitative measurement of polar head groups of phosphoglyceride in a body fluid, preferably urine.

By using the present invention in combination with metabolic and physiological measurements in or on nerve tissue, for instance by measuring metabolic changes in the CNS tissue by means of so-called magnetization transfer, it is now possible to determine and quantify the myelin degradation already present in the central nervous system. This analysis of demyelinization, the knowledge of the pathogenesis and the efficiency of therapies can greatly improve through use of the present invention. By using a biomarker according to the invention, it is thus now possible to monitor the actual demyelinization process.

The invention can be applied to mammals in general and to equines, bovines, porcines, ovines, myomorpha, canines, rodentia, simians and primates in particular. Preferably, the invention is applied to guinea pigs, dogs or humans.

The invention will be illustrated hereinbelow on the basis of an example.

### EXAMPLE 1

Sample Preprocessing

Prior to NMR spectroscopic analysis, 1 ml urine samples were lyophilized and reconstituted in 1 ml of sodium phosphate buffer (pH 6.0, based on D₂O) with 1 mM of sodium trimethylsilyl-[2,2,3,3,2H4]-1-propionate (TMSP) as an internal standard.

NMR Measurements

NMR spectra were recorded in triplicate in a fully automated manner on a Varian UNITY 400 MHz spectrometer provided with a proton NMR set-up and at a working temperature of 293 K. Free induction decays (FIDs) were collected as 64K data points with a spectral band width of 8,000 Hz; 45-degree pulses were used with a measurement time of 4.10 sec. and a relaxation delay of 2 sec. The spectra were determined by accumulation of 128 FIDs. The signal of the residual water was removed by a presaturation technique in which the water peak was irradiated at a constant frequency for 2 sec. prior to the measurement pulse.

The spectra were processed using the standard Varian software. An exponential window function with a line broadening of 0.5 Hz and a manual baseline correction was applied to all spectra.

After reference to the internal NMR standard (TMSP δ=0.0), line listings were compiled by means of the standard Varian NMR software. To obtain these line listings, all lines in the spectra with a signal intensity above the noise were collected and converted to a data file which was suitable for use of multivariate data analysis.

Determination of Metabolic Fingerprint or Difference Profile of Demyelinization Metabolites

By means of a 400 MHz NMR spectrometer, urine samples were tested of healthy individuals and of individuals in which demyelinization had been diagnosed. The spectra were processed and line listings were compiled by means of standard Varian software after reference to the internal standard. The NMR data reduction file was imported into Winlin VI. 10. Small variations of comparable signals in different NMR spectra were adjusted by using the Partial-Linear-Fit algorithm as described in WO 02/13228 and the lines were fitted without loss in resolution. The scale of the data was automatically adjusted and “normalized” to unit intensity. The endogenous and exogenous metabolites were eliminated from the NMR spectra, which led to the reduction of the data to specific and “significant” demyelinization-related changes. For this purpose, a threshold value was used by means of which 80-90% of the spectral line positions were eliminated.

A score plot of the NMR spectra was made by means of multivariate data analysis as described hereinabove. From the score plot, a metabolic fingerprint or difference profile of demyelinization was obtained by selecting rising and falling NMR signals with relatively high frequency of occurrence in urine of demyelinization patients. From these, a choice was made of approximately 35 NMR signals with a relevant contribution to demyelinization (regression>0.5). These NMR signals are shown in Table 1 and FIG. 2.

Other characteristic metabolites of which an increase in the concentration in urine could be determined and which could be identified as being involved in multiple sclerosis are given in Table 2.

TABLE 1
Characteristic increasing and decreasing NMR spectral
line positions due to demyelinization

| NMR spectral line | NMR spectral line |
|---|---|
| positions with | positions with |
| increasing values due | decreasing values due |
| to demyelinization | to demyelinization |
| in ppm ± 0.05 | in ppm ± 0.05 |
| 0.90 | 0.95 |
| 1.22 | 1.27 |
| 2.98 | 2.17 |
| 3.25 | 2.48 |
| 3.82 | 2.61 |
| | 2.93 |
| | 3.20 |
| | 3.50 |
| | 3.54 |
| | 3.56 |
| | 3.61 |
| | 3.81 |
| | 3.94 |
| | 3.96 |
| | 4.09 |
| | 4.10 |
| | 4.19 |
| | 7.00 |
| | 7.31 |

TABLE 2
Characteristic metabolites applicable
as biomarker(s) for multiple sclerosis

| Compound | Assignment | δ (ppm ± 0.05) | Multiplicity |
|---|---|---|---|
| N-acetylaspartate | CH3 | 2.05 | Singlet |
| | CH2 | 2.51 | Multiplet |
| | CH2 | 2.95 | Multiplet |
| Inositol | H1/H3 | 3.25 | Doublet doublet |
| | H2 | 4.10 | Triplet |
| Choline | NCH2 | 3.19 | Multiplet |
| | OCH2 | 3.94 | Multiplet |
| Neopterin | CH2 | 4.34 | Multiplet |
| | CH2 | 4.44 | Multiplet |
| | CH | 4.60 | Multiplet |
| | CH | 4.70 | Multiplet |
| | OCH2 | 5.20 | Singlet |
| Taurine | CH2SO3 | 3.26 | Triplet |
| | NCH2 | 3.31 | Triplet |
| Aliphatic region | | 0.50-3.50 | |
| Aromatic region | | 6.80-7.50 | |

1. A biomarker for detection of demyelinization, characterized in that the biomarker is a polar head group of phosphoglyceride.

2. A biomarker according to claim 1, wherein said polar head group is phosphatidylethanolamine, phosphatidylcholine, phosphatidyserine and/or phosphatidylinositol, or a part or derivate thereof.

3. A biomarker, comprising one or more metabolites or parts thereof chosen from the group consisting of N-acetylaspartate, inositol, choline, neopterin, and taurine, and combinations thereof.

4. Use of a biomarker according to claim 1, for monitoring demyelinization.

5. Use of a biomarker according to claim 1, for diagnosis and prognosis of multiple sclerosis.

6. A method for diagnosis and prognosis of demyelinization in a mammal, comprising measuring a biomarker according to claim 1 in a body fluid of an individual of the respective mammal.

7. A method according to claim 6, wherein said biomarker is measured by means of proton nuclear magnetic resonance spectroscopic analysis of metabolites in a body fluid and wherein said body fluid is urine.

8. A difference profile for the detection of demyelinization in a mammal, comprising a plurality of spectral line positions and optionally corresponding signal intensities of NMR spectral lines, which express the normalized difference between one or more NMR spectra of metabolites in a body fluid of one or more healthy individuals of said mammal, and one or more corresponding NMR spectra of metabolites in a corresponding body fluid of one or more individuals of said mammal in which demyelinization has been diagnosed.

9. A difference profile according to claim 8, wherein said mammal has been chosen from the group consisting of humans, dogs and guinea pigs.

10. A difference profile according to claim 8, wherein said body fluid is urine.

11. A difference profile according to claim 10, comprising the spectral lines and values corresponding thereto according to Table 1.

12. A method for the detection of demyelinization in a mammal, comprising the steps of providing an NMR spectrum of metabolites in a body fluid of an individual of said mammal in which demyelinization is suspected and comparing said NMR spectrum with a difference profile according to claim 8, determined for a corresponding body fluid.

13. A method according to claim 12, wherein said mammal has been chosen from the group consisting of humans, dogs and guinea pigs.

14. A method according to claim 12, wherein said body fluid is urine.

15. A method for manufacturing a difference profile for the detection of demyelinization in a mammal, comprising the steps of: a) providing a first set of positions and corresponding signal intensities of spectral lines in a NMR spectrum recorded from metabolites in a body fluid of one or more healthy individuals of said mammal; b) providing a second set of positions and corresponding signal intensities of spectral lines in a NMR spectrum recorded from metabolites in a corresponding body fluid of one or more individuals of said mammal in which demyelinization has been diagnosed; and c) detecting the differences between the normalized values of said first and second set, for obtaining said difference profile.

16. A method according to claim 15, wherein the determination of said normalized values comprises the use of the method according to WO 02/13228.

17. A method for identifying a biomarker for demyelinization, comprising manufacturing a difference profile according to claim 8 and identifying a metabolite which is characterized by one or more defined spectral lines in said difference profile.
